(19) United States
(12) Patent Application Publication
(10) Pub. No.: US 2003/0144676 A1
(43) Pub. Date: Jul. 31, 2003

(54) Anastomosis device and method

(76) Inventor: J. Kenneth Koster JR., Jacksonville, FL (US)

Correspondence Address: LAW OFFICE OF THOMAS C. SAITTA, 6821 SOUTHPOINT DR. NORTH, SUITE 203, JACKSONVILLE, FL, 32216, US

(21) Appl. No.: 10/056,636

(22) Filed: Jan. 25, 2002

(51) Int. Cl. A61B 17/08

(52) U.S. Cl. 606/155; 606/153

(57) ABSTRACT

The invention is a device for anastomosis of a vein graft to an aorta, wherein the device is used for creating a circular hole in the aorta wall, occluding the hole to prevent blood loss and providing a guide for insertion or placement of the vein into the hole in the aorta wall. The device has an elongated main body housing a punch mechanism, the punch mechanism having a disk-shaped punch head and a tubular cutting sleeve, which in conjunction act to remove a circular plug from the aorta wall, wherein the punch head is retractable relative to the cutting sleeve, with the cutting sleeve remaining in the aorta wall to prevent blood loss, and a lateral shaft having an internal bore communicating with the main bore of the cutting sleeve, whereby a vein graft can be introduced into the main bore and through the aorta wall.

### BACKGROUND OF THE INVENTION

This invention relates generally to the field of devices and methods used in performing anastomosis, i.e., the joining of a hollow or tubular organ to another hollow or tubular organ, and in particular to such devices used in the surgical joining of a vein graft to the aorta wall.

In coronary bypass surgery, a blocked or damaged segment of an artery is bypassed by attaching a vein graft to the aorta above the blocked point, such that blood flow is routed through the vein graft and around the blockage. In order to attach the graft, a hole must be created in the aorta wall, which may be formed by use of a scalpel but is preferably accomplished using a punch device in order to create a circular opening rather than a slit, as the circular opening is less likely to tear. The punch device typically comprises a sharp cone or bladed disk which creates a small slit in the aorta wall, through which the entire disk is passed. The disk is mounted onto a thin shaft, which is coaxially received by a tubular sleeve member, the end of which is provided with an annular cutting edge or rim. With the aorta wall now disposed between the disk and the sleeve, either the disk is withdrawn into the sleeve or the sleeve is advanced to surround the disk. This shearing operation cuts a circular opening in the aorta wall, and the plug cut from the wall is entrapped within the sleeve and disk when the punch device is removed.

It is necessary to temporarily occlude the opening in the aorta wall in some manner to prevent excessive loss of blood during the anastomosis procedure. In a most basic technique, the surgeon attempts to cover the opening with a finger, but this method is less than optimal. Use of the finger limits the surgeon's dexterity, as it is much easier to attach the vein graft if the surgeon has both hands free and does not need to concentrate on excessive blood loss. Alternatively, a surgical clamp may be applied across the aorta upstream of the opening, which reduces the necessity for the surgeon to rush through the attachment procedure and allows the surgeon free use of both hands, but this technique is problematic in that it stops all blood flow for the period of time necessary to complete the graft, and the pressure from the clamp may damage the aorta or may result in the release of plaque or other debris into the blood stream.

It is an object of this invention to provide an anastomosis device and a method of use for same. The device operates initially as a punch to create a well-defined circular opening in the aorta wall by removing a plug of wall material, then occludes the hole in the aorta wall after creation of the circular opening to prevent excessive blood loss during attachment of the vein graft, and subsequently provides a conduit for guidance of the vein to and into the hole in the aorta wall. In conjunction with a specialized anchoring means affixed to the end of the vein graft, the vein is inserted into the hole in the aorta wall and permanently connected without the need for extraneous suturing. These objects as well as other objects of the invention not expressly set forth above will be disclosed in the description to follow.

### SUMMARY OF THE INVENTION

The invention is a method and system for anastomosis of a vein graft to an aorta comprising a novel instrument or device which creates a circular hole in the aorta wall, occludes the hole to prevent blood loss and provides a guide for insertion of placement of the vein into the hole in the aorta wall. The device comprises in general an elongated main body defining a main bore and housing a punch mechanism, the punch mechanism comprising a disk-shaped punch head and a tubular cutting sleeve, which in conjunction act to create, remove and retain a circular plug from the aorta wall, means to retract the punch head relative to the cutting sleeve such that the cutting sleeve remains in the aorta wall to prevent blood loss, an obliquely connected lateral shaft having an internal bore communicating with the main bore of the cutting sleeve, and means to retract the punch head and a portion of the cutting sleeve in the proximal direction to expose the lateral shaft opening into the main bore, whereby a vein graft can be introduced into the main bore and through the aorta wall by passing the graft through the lateral shaft. A portion of the cutting sleeve remains disposed in the aorta wall to prevent blood loss during the insertion of the vein graft. The anchoring means for the vein graft preferably comprises an expandible annular wire lattice having short radial projections to secure the lattice in the interior of the vein and longer, flexible prongs which are compressed against the outer wall of the vein while resident within the lateral shaft and during passage through the cutting sleeve portion of the device, but which automatically expand radially to prevent withdrawal of the vein from the aorta wall after the anchoring means is fully inserted and extended from the distal end of the cutting sleeve. The anchoring means is secured to the vein prior to insertion into the lateral sleeve, and is advanced through the lateral shaft and cutting sleeve bore by use of a balloon catheter.

### BRIEF DESCRIPTION OF THE DRAWINGS

FIG. 1 is an exterior view of the anastomosis device.

FIG. 2 is a cross-sectional view of the anastomosis device inserted into the aorta wall, showing the vein graft in position in the lateral shaft.

FIGS. 3 and 4 show the punch mechanism in action forming the hole in the aorta wall.

FIGS. 5 through 7 show the method for securing the vein to the aorta wall using the anchoring means.

FIG. 8 is a view similar to FIG. 2 showing separation of the housing for retraction of the punch assembly after the hole has been created in the aorta wall.

### DETAILED DESCRIPTION OF THE INVENTION

With reference to the drawings, the invention will now be described in detail with regard for the best mode and the preferred embodiment. In general, the device comprises means to produce an opening in an aorta wall, means to seal the opening during the anastomosis process to preclude excessive blood loss, and means to receive and advance a vein graft through the device for attachment of the vein graft to the aorta wall.

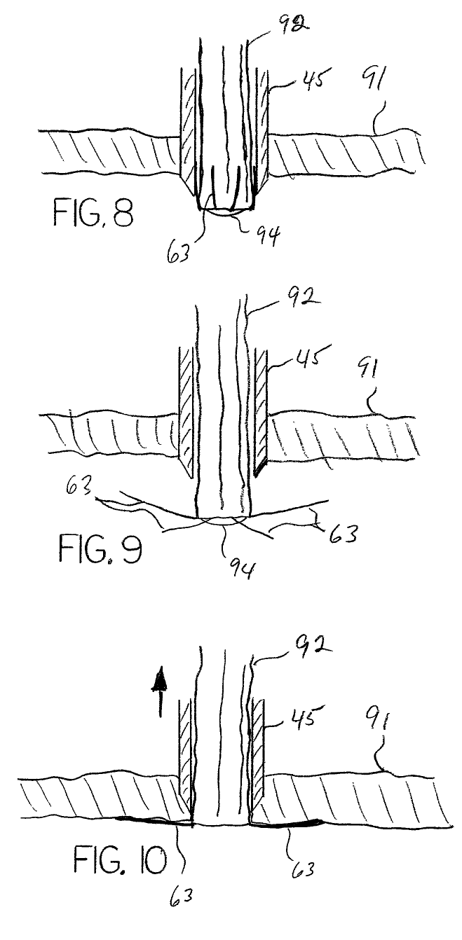

As shown in FIGS. 1 through 4 and 8, the anastomosis device 100 comprises an elongated housing 11 having a central bore 12 which coaxially receives a cutting sleeve assembly 40 and a punch assembly 30, where the cutting sleeve assembly 40 and the punch assembly 30 are movable in the axial direction relative to the housing 11. The housing 11 is divided into a proximal portion 14 and a distal portion 15 to define a punch retraction means 13, shown as comprising the proximal and distal portions 14 and 15 joined by connection means 19, such as a male and female threading or a key and slot combination, such that rotation of one portion relative to the other allows the two portions 14 and 15 to be separated, such that proximal portion 14 along with punch assembly 30 and the proximal portion 43 of cutting sleeve 45 can be withdrawn relative to the distal portion 15 and the distal portion 44 of cutting sleeve 45, as seen in FIG. 11. A sleeve advancement means 16, shown as a rachet mechanism 17, allows the cutting sleeve 45 to be advanced relative to the punch head 34 to create a hole in the aorta wall 91. A lateral shaft 50 is joined at an acute angle to the housing 11, the lateral shaft 50 having a lateral bore 51 covered on one end by a removable cap 52 and open on the other end such that it communicates with the central bore 12 of housing 11 when the proximal portion 43 of sleeve 45 is withdrawn along with the punch assembly 30.

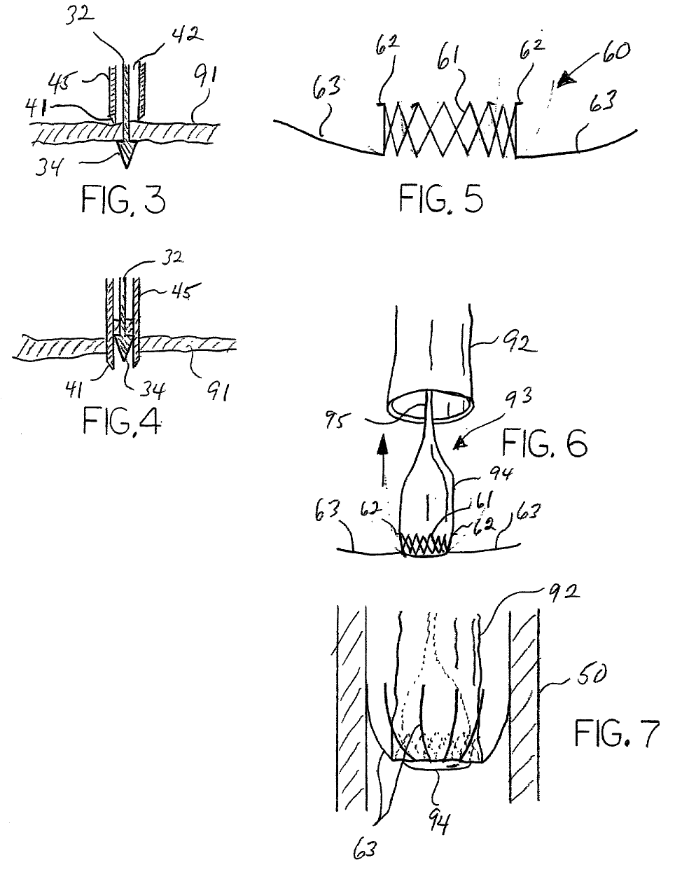

The punch assembly 30 comprises a shaft 33 bounded on the proximal end by a handle 33 and on the distal end by a punch head 34, the punch head 34 having a pointed or bladed distal portion 34a formed with a disk-shaped proximal end 34b. The cutting sleeve assembly 40 comprises a tubular cutting sleeve 45 defining an axial bore 42 with an annular cutting rim 41 which acts in cooperation with punch head 34, as shown in FIGS. 3 and 4, to cut a hole into the aorta wall 91 by removing a plug of wall material, the plug being retained within the cutting sleeve 45 behind the proximal end 34b of punch head 34. The cutting sleeve 45 comprises a separable proximal portion 43 and a distal portion 44. A spring member 46 maintains the cutting sleeve 45 in the retracted position until it is depressed by the surgeon to advance the annular cutting rim 41 past the punch head 34. After the hole has been made in the aorta wall 91, the distal portion 15 and the proximal portion 14 of the housing 11 are separated and the punch assembly 30 and sleeve proximal portion 43 are retracted, such that the punch head 34 and distal end of the sleeve proximal portion 43 are now positioned proximal to the opening for the lateral shaft 50, such that the lateral bore 51 now freely communicates with the central bore 12 of housing 11 and the axial bore 42 of sleeve distal portion 44, as shown in FIG. 11. The sleeve distal portion 44 remains disposed in the aorta wall 91 to prevent blood loss through the opening during the vein grafting procedure.

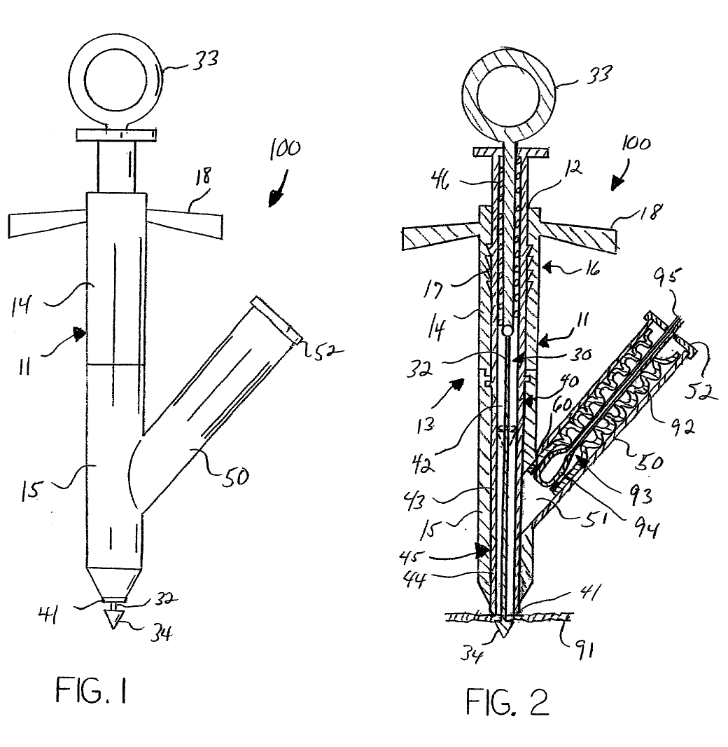

Preferable vein anchoring means 60 is shown in greater detail in FIGS. 5 through 7, and comprises an annular lattice or lattice ring of meshed or woven wire which is secured within the vein 92. The lattice ring initially occupies a configuration of minimal diameter which is expanded radially to present a larger diameter by action of a balloon catheter 93. To affix the lattice ring, it is placed onto the deflated head 94 of a balloon catheter 93, which is inserted into the vein 92 such that the lattice ring is positioned at the end of the vein 92 to be grafted, the body 95 of the catheter extending out the other end of the vein 92. The balloon head 94 is inflated to expand the lattice radially to embed it into the interior wall of the vein 92. The vein 92, with embedded anchor means 60 and the catheter head 94, is inserted into lateral shaft 50, with the catheter body 95 extending out through cap 52, as shown in FIG. 2. The anchor means 60 further comprises anchoring prongs 63, which are connected to the distal end of the lattice ring to extend generally radially outward. The anchoring prongs 63 are sufficiently flexible to allow them to be bent backward against the outer wall of the vein 92 when it is inserted into lateral shaft 50, yet retain enough elastic memory such that they will resume the radial configuration when not so confined.

The vein 92 is connected to the aorta wall 91 as shown in FIGS. 3 through 7. The circular opening in the aorta wall 91 has been formed by the surgeon first making a small incision and then inserting the punch head 34 through the incision and the aorta wall 91. Alternatively, the punch head 34 itself may be used to breach the aorta wall 91. The cutting sleeve assembly 40 is then advanced so that the annular cutting rim 41 slices through the aorta wall 91 in conjunction with the disk-shaped distal end 34b of the punch head 34 to remove a circular plug of wall material from the aorta wall 91. The housing 11 is separated into the distal portion 15 and the proximal portion 14, and the punch assembly 30 and proximal portion 43 of sleeve 45 are retracted to open the passageway to the lateral bore 51. The end of the vein 92 is now advanced by pushing the catheter body 95 into the lateral shaft 50. The catheter head 94, anchoring means 60 and vein 92 advance into the central bore 12, through the axial bore 42 of cutting sleeve distal portion 44 into the interior of the aorta wall 91. When the free ends of the anchoring prongs 63 clear the annular cutting rim 41, they spring outward to extend radially. The catheter head 94 is retracted slightly such that the prongs 63 abut the interior side of the aorta wall 91, and is then fully inflated to insure that the lattice ring is securely embedded and maximumly expanded to present the largest possible flow opening through the vein. The catheter head 94 is deflated and withdrawn from the vein 92 and from the lateral shaft 50. The entire device 100 is then withdrawn from the aorta wall 91, such that the entire vein 92 passes through the distal end of the device and remains attached to the aorta wall 91.

Alternatively to the complete method set forth above where both the anastomosis device 100 and the anchoring means 60 are utilized to secure the vein 92 to the aorta wall 91, it is contemplated that the device 100 could be used with other methods for securing the vein 92, such as suturing, stapling or with the use of other types of anchor devices.

It is understood that equivalents and substitutions for certain elements set forth above may be obvious to hose skilled in the art, and thus the true scope and definition of the invention is to be as set forth in the following claims.

I claim:

1. An anastomosis device for joining a vein graft to an aorta wall, comprising:
means to produce an opening in an aorta wall, said means comprising in combination a punch assembly and a cutting sleeve assembly disposed within a central bore of a housing;
a lateral shaft obliquely connected to said housing, said lateral shaft having a lateral bore communicating with said central bore;
wherein said lateral shaft is adapted to receive a vein graft such that said vein graft may be advanced through said lateral bore, said central bore and said opening in said aorta wall for joining to said aorta wall.

2. The device of claim 1, further comprising means to seal said opening in said aorta wall after said opening is produced and during said advancement of said vein graft in order to prevent loss of blood.

3. The device of claim 2, wherein said cutting sleeve comprises a distal portion and a proximal portion, and wherein said punch assembly and said proximal portion of said cutting sleeve is retractable, and wherein said lateral bore freely communicates with said central bore when said punch assembly and said proximal portion of said cutting sleeve is retracted.

4. The device of claim 3, wherein said punch assembly and said portion of said cutting sleeve retain a portion of said aorta wall removed to create said opening when said punch assembly and said proximal portion of said cutting sleeve are retracted.

5. The device of claim 4, wherein said means to seal said opening in said aorta wall comprises said distal portion of said cutting sleeve.

6. The device of claim 5, said cutting sleeve assembly further comprising means to advance said cutting sleeve relative to said punch assembly for producing said opening in said aorta wall.

7. The device of claim 6, wherein said housing comprises a proximal portion and a distal portion, said proximal portion being separable from said distal portion and retractable in order to retract said punch assembly and said proximal portion of said cutting sleeve.

8. The device of claim 7, said lateral shaft further comprising a cap member adapted to receive a balloon catheter therethrough.

9. An anastomosis device for joining a vein graft to an aorta wall comprising:
an elongated housing comprising a central bore;
an annular cutting sleeve assembly disposed within said central bore, wherein at least a portion of said cutting sleeve assembly is retractable relative to at least a portion of said housing;
a retractable punch assembly disposed within said cutting sleeve assembly, wherein said punch assembly is retractable relative to at least a portion of said housing;
a lateral shaft obliquely joined to said housing and comprising a lateral bore;
wherein said retractable portion of said cutting sleeve assembly and said retractable punch assembly are retractable beyond said lateral bore such that said lateral bore is in communication with said central bore when said retractable portion of said cutting sleeve assembly and said retractable punch assembly are retracted, whereby a vein graft may be advanced through said lateral bore and said central bore.

10. The device of claim 9, said cutting sleeve assembly further comprising a distal portion and a proximal portion, such that only said proximal portion is retractable.

11. The device of claim 10, further comprising means to advance said cutting sleeve assembly relative to said punch assembly.

12. The device of claim 11, said housing further comprising a distal portion and a proximal portion, said proximal portion being separable from said distal portion.

13. The device of claim 12, further comprising a balloon catheter, and wherein said lateral shaft further comprises a cap member adapted to receive said balloon catheter therethrough.

14. A method of performing anastomosis of a vein graft to an aorta wall comprising the steps of:
providing an anastomosis device comprising a punch assembly having a punch head and a cutting sleeve disposed in a central bore, and a lateral shaft having a lateral bore communicating with said central bore, said lateral bore adapted to receive a vein graft;
inserting a vein graft into said lateral bore;
inserting said punch head into said aorta wall;
advancing said cutting sleeve to against said punch head to create an opening in said aorta wall;
retracting said punch head and at least a portion of said cutting sleeve proximally beyond said lateral bore, while leaving a portion of said anastomosis device disposed within said opening to prevent blood loss;
advancing said vein graft through said central bore and into said opening in said aorta wall;
securing said vein graft to said aorta wall; and
removing said anastomosis device from said opening and from said vein graft.

15. The method of claim 14, further comprising providing a balloon catheter within said lateral bore, whereby said vein graft is advanced into said opening by advancing said balloon catheter.

16. The method of claim 15, whereby said step of retracting said punch head and at least a portion of said cutting sleeve proximally beyond said lateral bore is accomplished by separating a proximal portion of said anastomosis device from a distal portion of said anastomosis device.
